(19) United States
(12) Patent Application Publication
(10) Pub. No.: US 2020/0397947 A1
(43) Pub. Date: Dec. 24, 2020

(54) Scaffold-Free Cell Sheet for Nerve Repair

(71) Applicant: UNIVERSITY OF PITTSBURGH-OF THE COMMONWEALTH SYSTEM OF HIGHER EDUCATION, Pittsburgh, PA (US)

(72) Inventors: Fatima Syed-Picard, Pittsburgh, PA (US); Tia Christine Calabrese, Pittsburgh, PA (US)

(21) Appl. No.: 16/976,821

(22) PCT Filed: Mar. 1, 2019
(86) PCT No.: PCT/US19/20226

§ 371 Date: Aug. 31, 2020

Related U.S. Application Data
(60) Provisional application No. 62/637,555, filed on Mar. 2, 2018.

(51) Int. Cl. A61L 27/36; A61L 27/38; C12N 5/0793; C12N 5/0775

(52) U.S. Cl. CPC A61L 27/3675; A61L 27/3633; A61L 27/383; C12N 2501/115; C12N 5/0619; C12N 5/0664; A61L 2430/32; A61L 27/3687

(57) ABSTRACT

A method of repairing nerve tissue is provided. A method of inducing neurite outgrowth in neurons also is provided.

### CROSS REFERENCE TO RELATED APPLICATIONS

This application claims the benefit of U.S. Provisional Patent Application No. 62/637,555, filed Mar. 2, 2018, which is incorporated herein by reference in its entirety.

Provided herein are methods of generating nerve tissue and repairing nerve tissue in a patient. Also provided are uses for neural crest-derived cells, such as dental pulp cells, for example, for use in the preparation of, or regeneration of nerve tissue. In aspects and embodiments, the methods and cells induce or produce neurite outgrowth in neurons.

Peripheral nerve damage is a commonly encountered clinical problem caused by trauma, disease, or surgical injury. The current gold standard treatment utilizes autologous nerve grafts; however, this requires a prolonged repair time and full functional recovery is not achieved.

Current methods of treatment for defects of the facial nerve smaller than 1 cm include direct end-to-end reconstruction where the proximal and distal stumps are directly reconnected, and larger defects are bridged using autograft tissue. In both of these approaches, nerve regeneration can take up to 18 months and complete functional recovery is not achieved. The long recovery times associated with these procedures and permanent sequelae are extremely frustrating to patients. One factor contributing to these results is diminished Schwann cell support. These cells facilitate nerve regeneration by providing neurotrophic factors (NTFs), which are known to promote neuron survival and repair. A major research endeavor for nerve regeneration is developing delivery methods of NTFs. However, the challenges with current NTF delivery materials is engineering a system that minimizes the initial burst release and can also facilitate the sustained release of the biomolecules. Alternatively, researchers are proposing delivering cells genetically modified to produce NTFs, however, these cells have been highly manipulated and therefore pose manufacturing and regulatory challenges for therapeutic translation.

There is a need for methods and reagents for repair of nerve damage, such as peripheral nerve damage.

### SUMMARY

In one aspect, a method of producing neurite outgrowth in a neuron is provided. The method comprises: culturing neural crest-derived cells, such as cell populations comprising neural crest-derived stem cells and/or neural crest-derived progenitor cells, to produce one or more neurotrophic factors; and exposing neural tissue comprising a neuron to one or more neurotrophic factors produced by the cultured neural crest-derived cells for a time sufficient to produce neurite outgrowth from a neuron, thereby producing neurite extension from one or more neurons of the neural tissue.

In another aspect, a method of repairing damaged nerve tissue in a patient is provided. The method comprises implanting cultured neural crest-derived cells, such as cell populations comprising neural crest-derived stem cells and/or neural crest-derived progenitor cells, adjacent to a site of nerve tissue damage in the patient, thereby causing neurite outgrowth in the nerve tissue.

### BRIEF DESCRIPTION OF THE DRAWINGS

FIG. 1: Fabrication of Dental pulp cell (DPC) sheet. (A) Microscopic image showing confluent DPC and (B) DPC forming cell sheet, arrow pointing to the cell sheet that is detaching from the base of a well from 6 well plate dish. (C) Photograph of DPC sheet being handled by forceps in a 6 well plate dish.

FIG. 2: Histological characterization of DPC sheets. (A) & (B) H&E and immunofluorescent staining of DPC sheets cultured in media without Fibroblastic growth factor (FGF2) respectively. (D) & (E) DPC sheets cultured in media with FGF2. (C) & (F) Negative controls for immunofluorescent staining of type 1 collagen for cell sheets cultured in without & with FGF2 respectively. H&E staining of DPC sheet sections show that the cell sheets are solid and multicellular structures. Immunofluorescent staining shows that the extracellular matrix of DPC sheet contains type 1 collagen (green in original). Cell nuclei was detected by DAPI stain (blue in original).

FIG. 3: Quantification of cell number in DPC sheet. Bar graph shows that number of cells in DPC sheets cultured in media with FGF2 have approximately twice the number of cells when compared with DPC sheets cultured in media without FGF2. P value <0.05 was considered significant. FGF2: Fibroblastic growth factor 2.

FIG. 4: Reverse transcription-polymerase chain reaction (RT-PCR) results show that dental pulp cells cultured as cell sheets express genes for (A) Brain derived neurotrophic factor (BDNF), (B) Glial-cell derived neurotrophic factor (GDNF) and (C) Neurotrophic factor 3 (NT3), their expression was increased in the group treated with FGF2. RNA extracted from the DPC used at the time of plating wells was used as control. Fold change expressed as mean +/−standard deviation with P value <0.05 considered as significant. FGF2: Fibroblastic growth factor 2.

FIG. 5: Enzyme linked immunosorbent assay (ELISA) was done to detect the concentrations of Brain derived neurotrophic factor (BDNF), Glial-cell derived neurotrophic factor (GDNF) and Neurotrophic factor 3 (NT3) proteins in conditioned media collected from DPC sheets cultured in +/−FGF2 and uncultured growth media +/−FGF2 was used as negative control. (A) & (B) shows that the amount of proteins BDNF & GDNF secreted was high in CM+FGF2 compared to CM-FGF2. (C) Shows that NT3 protein secretion was increased in CM-FGF2 compared to CM+FGF2. Concentrations expressed as means +/−standard deviations with P value <0.05 considered as significant. GM+/−FGF2: Growth media +/−fibroblastic growth factor 2; CM+/−FGF2: Conditioned media +/−fibroblastic growth factor 2.

FIG. 6: Neurite extension in SH-SY5Y neurons was detected by beta-3 tubulin immunostaining (red in original) and counterstained with DAPI (blue in original) for the nuclei. Neurite extension in SH-SY5Y neurons was induced by culturing in conditioned media +/−FGF2 and growth media +/−FGF2. (A) & (B) SH-SY5Y neurons cultured in growth media without and with FGF2, (C) & (D) SH-SY5Y neurons cultured in conditioned media without and with FGF2 respectively.

FIG. 7: Neurite inhibitory assay. To validate the neurite extension effect induced by the NTF in conditioned media, SH-SY5Y neurons were cultured with conditioned media supplemented with inhibitors for NTF. TrKB, TrKC receptor blockers & antibody to GDNF were added to the conditioned media and growth media and neurite quantification was done. (A) Bar graph shows percentage of neurite positive SH-SY5Y cells and (B) range of neurite length extensions of SH-SY5Y neurons after culturing in media without and with inhibitors. Note: Extensions of SH-SY5Y cells twice the length of cell body were considered as neurite and length of longest neurite per neuron was measured. GM+/−FGF2: Growth media +/−fibroblastic growth factor 2. CM+/−FGF2: Conditioned media +/−fibroblastic growth factor 2.

FIG. 8: Crush nerve defect and DPC sheet implantation in rats. (A) Crush defect of 3 mm was induced in the buccal branch of the facial nerve in immunocompromised rats with a non-serrated forceps for 20 seconds. (B) DPC sheet was implanted at the site of crush defect and secured with 9-0 nylon suture. (C) 4 weeks post-surgery harvesting the regenerated nerve, arrow points to DPC sheet that can be seen around the regenerated nerve.

FIG. 9: Histological evaluation of the regenerated nerve. About 3 mm of the buccal branch of the facial nerve was crushed in rats and treated with DPC sheet implantation in the experimental group and without any treatment in the control group. (A) Hematoxylin and eosin stain and (D) immunostaining for axons (green in original) and DAPI for nuclei (blue in original) of a normal nerve as a positive control. (B, C) H&E stained images of the untreated crushed nerve and crushed nerve with DPC sheet implantation. (E) Immunostaining done with anti-beta tubulin stain (green in original) and counterstained with DAPI (blue in original) for nuclei in the untreated crush nerve showed cellular infiltration at the injured site and the axon extension to be discontinuous (G) Higher magnification image of boxed region seen in (E). (F) Crush nerve implanted with DPC sheets shows axon extensions to be more continuous across the defect. (H) Higher magnification image of boxed region in (F). The black and white arrows in (C), (F) and (H) point to the presence of DPC sheet around the nerve in the H&E and immunostained images respectively. The delineation shows the nerve trunk surrounded by DPC cell sheet in the animals treated with DPC sheet implantation.

### DETAILED DESCRIPTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include, but are not limited to embodiments “consisting essentially of” and “consisting of” these stated elements or steps. For definitions provided herein, those definitions refer to word forms, cognates and grammatical variants of those words or phrases.

As used herein, the terms “patient” or “subject” refer to members of the animal kingdom including but not limited to human beings and “mammal” refers to all mammals, including, but not limited to human beings.

As used herein, “treatment” or “treating” of a wound or defect means administration to a patient by any suitable dosage regimen, procedure and/or administration route of a composition, device or structure with the object of achieving a desirable clinical/medical end-point, including, for example, attracting progenitor cells, healing a wound, correcting a defect, or repairing a nerve.

As used herein, the terms “cell” and “cells” refer to any types of cells from any animal, such as, without limitation, rat, mouse, monkey, and human. For example and without limitation, cells can be progenitor cells, e.g. pluripotent cells, including stem cells, induced pluripotent stem cells, multi-potent cells, or differentiated cells, such as endothelial cells and smooth muscle cells. “Cells” also includes populations of cells, such as, for example, a population of cells produced by culturing dental pulp or cells from other neural crest-derived tissue. In certain aspects, cells for medical procedures can be obtained from the patient for autologous procedures, or from other donors for allogeneic procedures, or from xenogeneic sources.

Provided herein is a scaffold-free cell structure, such as a sheet, comprising minimally manipulated neural crest-derived cells, such as dental pulp cells, that naturally express neurotrophic factors. Cell structures can be placed adjacent to a neuron to induce neurite outgrowth from the neuron. For example, cell sheets can be wrapped around peripheral nerves repaired following current standard surgical treatment methods to accelerate healing and enhance motor recovery. The cell structures, such as dental pulp cell sheets, could be used alone and wrapped around damaged nerve tissues or could be combined with engineered nerve conduit materials to form a hybrid device to bridge nerve gaps. In addition to dental pulp cells, cell sheets prepared from other neural crest-derived cells are expected to be useful for this purpose.

Neurotrophic factors (NTF) are proteins known to enhance axon regeneration and growth. Neural crest-derived tissue, for example, dental pulp tissue, contains a population of stem/progenitor cells (DPC) that secrete NTFs a characteristic likely due to their neural crest origin. Furthermore, these cells are easily accessible from autologous sources. The goal of this study was to develop and characterize scaffold-free cell sheets as a NTF delivery system. We hypothesize that, neural crest-derived cell sheets, such as dental pulp cell sheets will express NTFs including brain-derived neurotrophic factor (BDNF), glial cell line-derived neurotrophic factors (GDNF) and neurotrophin-3 (NT-3), and will accelerate repair of damaged nerves and improve functional recovery.

Cells useful for nerve tissue repair include cell populations obtained from culturing cells obtained from neural crest-derived tissue (tissue arising from the neural crest), which, among other tissues, include many of the skeletal and connective tissue of the head, or the cranial mesenchyme, such as cartilage, bone, cranial neurons, glia, and connective tissues of the face. Neural crest cells enter the pharyngeal arches and pouches to give rise to thymic cells, odontoblasts of the tooth primordia, and the bones of middle ear and jaw. Specific examples of neural crest-derived tissue include, without limitation, dental pulp cells, dental pulp stem cells, and cells obtained from dental tissue, periodontal ligament tissue, apical papilla, and corneal stroma. Methods of identifying, isolating and preparing cells, including populations of cells comprising stem cells and induced stem cells, are broadly-known. Depending on the manner or use of the cells, autologous, allogeneic, or xenogeneic cells may be used to produce NTFs and/or cell structures according to in any aspect or embodiment of the methods described herein.

A “neuron”, or alternatively a “nerve cell”, is a cell of the central nervous system, or peripheral nervous system of an animal, such as a human, that conducts nerve impulses. Neurons can be multipolar, bipolar, unipolar, or pseudounipolar, and include, without limitation, sensory (afferent) neurons, motor (efferent) neurons, association neurons, projection neurons, intrinsic neurons (interneurons), Purkinje cells, pyramidal cells, olifactory cells, retinal cells, and ganglion cells, among many others. Neural tissue is tissue that comprises one or more neurons. A “neurite” (also, “neuronal process”) is a projection from the cell body of a neuron, such as an axon or dendrite. Neurite outgrowth, characterized by neurite extension, is a characteristic of growth of neuronal development, and in the context of one aspect of the present disclosure, neuronal growth and repair.

A “cell growth scaffold” is a mesh, matrix, particle, surface, or other material upon which or into which a cell can be deposited and can be maintained in a living state, and often propagates (multiplies) in the presence of suitable cell growth media.

As used herein, the term “polymer composition” is a composition comprising one or more polymers. As a class, “polymers” includes, for example and without limitation, homopolymers, heteropolymers, copolymers, block co-polymers and can be both natural and synthetic. Homopolymers contain one type of building block, or monomer, whereas copolymers contain more than one type of monomer. Polymers can have any shape, including, without limitation: linear, branched, star-shaped, comb-shaped, and dendrimeric.

A polymer “comprises” or is “derived from” a stated monomer if that monomer is incorporated into the polymer. Thus, the incorporated monomer that the polymer comprises is not the same as the monomer prior to incorporation into the polymer, in that at the very least, during incorporation of the monomer, certain groups, e.g., terminal groups, that are modified during polymerization are changed, removed, and/or relocated, and certain bonds may be added, removed, and/or modified. A monomer may be a “macromer”, an oligomer or polymer that is the combination product of two or more smaller residues, and is employed as a monomer in preparation of a larger polymer. An incorporated monomer is referred to as a “residue” of that monomer. A polymer is said to comprise a specific type of linkage if that linkage is present in the polymer, thus, a polyester comprises a plurality of ester linkages, a polyurethane comprises a plurality of urethane (e.g., carbamate) linkages, and a poly(ester urethane) urea comprises ester, urethane, and urea linkages. Unless otherwise specified, molecular weight for polymer compositions refers to weight average molecular weight (Mw). Composition of a copolymer may be expressed in terms of a ratio, typically a molar ratio, of incorporated monomers or as a feed ratio of monomers prior to polymerization. In the case of feed ratios, the relative amount of each monomer incorporated into the copolymer is influenced by reaction kinetics, and the nature of the chemical reaction(s) employed to join the monomers.

A “moiety” is a portion of a molecule, compound or composition, and includes a residue or group of residues within a larger polymer.

A bioerodible polymer is a polymer that degrades in vivo over a time period, which can be tailored to erode over a time period ranging from days to months, and up to two years. For example, a polymeric structure, when placed in vivo, will degrade within a time period of up to two years. By “bioerodible,” it is meant that a polymer, once implanted and placed in contact with bodily fluids and/or tissues, will degrade either partially or completely through chemical, biochemical and/or enzymatic processes, as compared to non-bioerodible polymers. Non-limiting examples of such chemical reactions include acid/base reactions, hydrolysis reactions, and enzymatic cleavage. In certain aspects, the biodegradable polymers may comprise homopolymers, copolymers, and/or polymeric blends comprising, without limitation, one or more of the following monomers: glycolide, lactide, caprolactone, dioxanone, and trimethylene carbonate. In other non-limiting embodiments, the polymer(s) comprise labile chemical moieties, non-limiting examples of which include esters and anhydrides, which can be useful in, for example and without limitation, controlling the degradation rate of the devices described herein and/or the release rate of therapeutic agents from a cell growth scaffold or particles.

A cell growth scaffold is a structure, such as a porous matrix or mesh formed from any suitable composition(s), typically including polymer(s), upon which cells can be maintained in a living state or propogated.

A composition is “free” of a stated constituent if that constituent is not present in the composition or is present in insubstantial amounts that do not interfere, or that insignificantly interfere, with intended use and function of the composition.

By “biocompatible,” it is meant that a composition or device, e.g., a polymer composition or a cell growth scaffold, and its normal in vivo degradation products are cytocompatible and are substantially non-toxic and non-carcinogenic in a patient within useful, practical, and/or acceptable tolerances. By “cytocompatible,” it is meant that a composition, device, polymer, cell growth scaffold, etc. can sustain a population of cells and/or the polymer composition, device, and degradation products thereof are not cytotoxic and/or carcinogenic within useful, practical, and/or acceptable tolerances. For example, the polymer when placed in a human epithelial cell culture does not adversely affect the viability, growth, adhesion, and number of cells. In one non-limiting aspect, the compositions and/or devices are “biocompatible” to the extent they are acceptable for use in a human or veterinary patient according to applicable regulatory standards in a given jurisdiction. In another example, the biocompatible polymer, when implanted in a patient, does not cause a substantial adverse reaction or substantial harm to cells and tissues in the body, for instance, the polymer composition or device does not cause unacceptable inflammation, allergic reaction, necrosis, or an infection resulting in harm to tissues from the implanted scaffold.

Compositions may comprise a pharmaceutically acceptable carrier, or excipient. An excipient is an inactive substance used as a carrier for the active ingredients of a medication. Although “inactive,” excipients may facilitate and aid in increasing the delivery, stability or bioavailability of an active ingredient in a drug product. Non-limiting examples of useful excipients include: anti-adherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

A living cell structure or sheet is provided that can be used as a NTF delivery system that can augment current facial nerve surgical treatment modalities to accelerate healing and improve functional recovery. These cell sheets comprise only the cells and their extracellular matrix. Since neural crest-derived cells (cells that originate from the embryonic neural crest, for example, dental pulp cell populations, for example and without limitation, including dental pulp progenitor or stem cells, endogenously express NTFs, the cells are minimally manipulated for forming the described cell structure, such as a cell sheet). These cell structures, such as sheets, would supplement current surgical techniques by being wrapped around or placed adjacent to a defect region following procedures including nerve anastomosis or the insertion of autograft tissue. This technology is a simple and efficient manner to provide sustained NTF delivery to nerve tissue defects in congruence with current surgical treatments. Through the delivery of NTFs, neural crest-derived cell sheets would enhance axon regeneration compared to the current standard of care resulting in reduced regeneration time and improved functional outcomes.

Neural crest-derived cell structures such as dental pulp cell sheets meet all of the criteria of the ideal material for nerve repair. Ideal materials for nerve repair are biocompatible, are easily prepared and customizable to the size of the defect, and include bioactivity to promote axonal regeneration (Gaudin R, et al. Approaches to Peripheral Nerve Repair: Generations of Biomaterial Conduits Yielding to Replacing Autologous Nerve Grafts in Craniomaxillofacial Surgery. Biomed Res Int. 2016; 2016:3856262. Epub 2016/08/25. doi: 10.1155/2016/3856262. PubMed PMID: 27556032; PMCID: PMC4983313). Cell structures, such as cell sheets can be formed using autologou, allogeneic or xenogeneic cells, and they can be easily scaled up or down to fit defect size, and the cell structures are robust and can be easily handled by the surgeons for implantation. Furthermore, the cell structures express NTFs endogenously and improve axon growth. This technology addresses a current unmet clinical need to shorten regeneration time and improve functional outcomes for nerve, e.g., facial nerve, repair.

The scaffold-free cell structures e.g., sheets, are robust and can easily be handled by surgeons for implantation. The DPCs in these cell sheets express the genes for NTFs including brain-derived growth factor (BDNF), glial-cell derived neurotrophic factor (GDNF), and neurotrophin-3 (NT-3). Furthermore, DPC sheets have the functional effect of promoting neuritogenesis in SH-SY5Y human-derived neuronal cells in vitro.

In one aspect of the invention, a method of producing neurite outgrowth in a neuron is provided. The method may be performed in vitro or in vivo. The method comprises, first, culturing neural crest-derived cells to produce a solid tissue structure, such as a sheet or other shape. Then neural tissue comprising a neuron is exposed to one or more neurotrophic factors produced by the tissue structure for a time sufficient to produce neurite outgrowth from a neuron, thereby producing neurite extension from one or more neurons of the neural tissue. The neurons may be cultured directly on or adjacent to the solid tissue structure. In one aspect, the neurons are in a living nerve and the cell structure is implanted adjacent to or surrounding the nerve, for example, as a wrap for the nerve. Alternatively, the neurons are cultured in the same tissue culture vessel, e.g. in a bioreactor, flask, or plate, as are known in the art, such that neurotrophic factors produced by the cell structure or by neural crest-derived cells disperse in culture medium in which the neurons also are grown or cultured. In another aspect, conditioned culture medium is prepared by culturing the neural crest-derived cells, e.g., dental pulp cells, in cell culture medium to produce conditioned media and then using the conditioned media to culture neurons, e.g., as an additive to fresh culture media, or to otherwise apply the conditioned media to neurons to stimulate neurite outgrowth.

In another aspect, a method of repairing nervous tissue damage in a patient is provided. Nerve damage can be from trauma, disease, or surgical injury, and can be a peripheral nerve, or CNS nerve tissue, such as spinal cord tissue. The method comprises implanting a living structure, such as a sheet of cultured, e.g., dental pulp cells, or other cells, stem cells, and/or progenitor cells isolated from neural crest-derived tissue (“neural crest-derived cell”), adjacent to a nerve injury in patient. Although autologous cells may be preferred in instances, allogeneic or xenogeneic cells may be used. In one aspect, the structure, e.g., sheet, is formed by ex vivo culturing of the neural crest-derived cells, such as a population of cells derived from the neural crest and comprising, e.g., progenitor and stem cells, for example and without limitation, dental pulp cells, typically to confluence or post-confluence, in order to produce a sufficiently robust extracellular matrix ex vivo. The nerve injury may be, for example and without limitation, a facial nerve injury, an injury to any peripheral nerve, or a spinal cord injury. The method also optionally comprises obtaining neural crest-derived cells, such as a population of cells derived from the neural crest and comprising, e.g., progenitor and stem cells, for example and without limitation dental pulp cells, e.g., from the patient and culturing the cells to confluence or to post-confluence. In one aspect, the area of nerve damage is wrapped in a cell sheet, e.g., of cultured autologous dental pulp cells. In one aspect, the nerve damage is from trauma. In another aspect, the nerve damage is from disease. In another aspect, the nerve damage is from surgical injury, or from surgical repair, such as an anastamosed peripheral nerve or a facial nerve that is surgically repaired after trauma or disease.

In a further aspect, the living structure, such as a sheet, of cultured cells, e.g., autologous, dental pulp cells, or other cells, stem cells, or progenitor cells isolated from neural crest-derived tissue, is implanted with a nerve guide or nerve conduit, as are broadly-known, which are devices supportive of nerve growth. Nerve guides are available commercially, for example, and without limitation, NeuraGen® (Integra LifeSciences) or Neurawrap™ (Integra LifeSciences), NeuroMatrix™, Neuroflex™, or Neuromed™ (Stryker), GEM Neurotube® (Synovis), Neurolac® (Polyganics), AxoGuard® (Axogen, Inc.), and Surgisis® nerve cuff (Cook).

In yet another aspect, the living structure, such as a sheet, of cultured cells, e.g., dental pulp cells, or other cells, stem cells, or progenitor cells isolated from neural crest-derived tissue, is implanted with a cell growth scaffold, such as a device formed from a suitable polymer or decellularized tissue, such as an ECM material.

Dental pulp cells are typically isolated from extracted teeth, such as from third molars, supernumerary teeth, deciduous teeth, or teeth removed for orthodontic purposes. In aspects, pulp tissue is removed, and treated with a protease, such as a collagenase or dispase. Cells are separated from debris, e.g., by centrifugation or using a cell strainer, and are cultured in a culture dish in any suitable cell culture medium, as are broadly known, capable of supporting growth of dental pulp cells (hereinafter referred to as “dental pulp stem cell culture medium”). Culture medium useful for growth and expansion of mesenchymal stem cells can be used for growth of dental pulp stem cells. In one aspect, for example and without limitation, dental pulp culture medium consists of: 80% DMEM high glucose with Glutamax (Gibco), 20% fetal bovine serum (Atlanta Biologicals), and 100 U/ml penicillin and streptomycin (Gibco). Cell culturing medium is broadly-available from a multitude of sources. Stem cell culturing products are commercially available from a number of sources, including from Stemcell Technologies, Inc. of Cambridge, Mass. In one aspect, ascorbic acid is added in order to strengthen the integrity of the formed sheet. In another aspect fibroblast growth factor 2 (FGF2) is added, for example, in the range of, for example, 1 ng to 50 ng per 2.5 mL of culture medium.

To form a solid tissue structure, such as a cell sheet, cells are grown under suitable conditions, for example, past confluence, until a sheet or other solid structure is formed having sufficient mechanical strength for handling. The cells plus the extracellular matrix produced by the cells form a living structure, such as a living sheet (“cell sheet”), that can be removed from the culture dish, physically manipulated, and implanted into a patient adjacent to a nerve, e.g., by suturing or gluing with, e.g., fibrin glue, as is broadly-known in the medical arts. Any suitable tissue culture dish or surface can be utilized to produce the cell structures or sheets, such as petri dishes, multi-well plates (e.g., a 6-well tissue culture plate as used below in the Example), flasks, or other surfaces. Cell sheets can be cultured on microscope slides within a tissue culture flask or any other suitable surface. Surfaces of different stiffness or elasticity can be used to grow the cell sheet. In aspects, the surface on which the cell sheet is grown or produced can be coated with a cell adhesion composition, such as laminin or fibronectin, as are known in the cell-culturing arts.

Prior to use, the cell structure or sheet may be washed to remove cell culture medium components, such as xenogeneic serum, antibiotics, and any other undesirable constituents. The cell sheet may be washed in any suitable solution, such as serum-free medium, phosphate-buffered saline, or normal (0.9% w/v) saline, optionally including suitable antibiotics.

In one aspect, the cell structure or sheet according to any aspect described herein is implanted at a site of nerve tissue damage with a cell growth scaffold, such as porous or fibrous matrices of synthetic or natural bioerodible polymers, non-erodible polymers, ECM products, such as an ECM material, such as decellularized, nuclease-treated tissue, or a gel prepared therefrom, or hydrogels comprising natural or synthetic polymer compositions.

Non-limiting examples of a bioerodible polymer useful for cell growth scaffolds, hydrogels, or particles include: a polyacrylate or polymethacrylate, a polyacrylamide or polymethacrylamide, a polyester, a polyester-containing copolymer, a polyanhydride, a polyanhydride-containing copolymer, a polyorthoester, and a polyorthoester-containing copolymer. In one aspect, the polyester or polyester-containing copolymer is a poly(lactic-co-glycolic) acid (PLGA) copolymer. In another embodiment, the bioerodible polymer is selected from the group consisting of poly(lactic acid) (PLA); poly(trimethylene carbonate) (PTMC); poly(caprolactone) (PCL); poly(glycolic acid) (PGA); poly(glycolide-co-trimethylenecarbonate) (PGTMC); poly(L-lactide-co-glycolide) (PLGA); polyethylene-glycol (PEG-) containing block copolymers; and polyphosphazenes. Additional bioerodible, biocompatible polymers include: a poly(ester urethane) urea (PEUU); poly(ether ester urethane)urea (PEEUU); poly(ester carbonate)urethane urea (PECUU); poly(carbonate)urethane urea (PCUU); a polyurethane; a polyester; a polymer comprising monomers derived from alpha-hydroxy acids such as: polylactide, poly(lactide-co-glycolide), poly(L-lactide-co-caprolactone), polyglycolic acid, poly(dl-lactide-co-glycolide), and/or poly(1-lactide-co-dl-lactide); a polymer comprising monomers derived from esters including polyhydroxybutyrate, polyhydroxyvalerate, polydioxanone, and/or polyglactin; a polymer comprising monomers derived from lactones including polycaprolactone; or a polymer comprising monomers derived from carbonates including polycarbonate, polyglyconate, poly(glycolide-co-trimethylene carbonate), or poly(glycolide-co-trimethylene carbonate-co-dioxanone).

Non-limiting examples of natural bioerodible polymers useful for tissue growth scaffolds, hydrogels, or particles include proteins, glycosaminoglycans, and polysaccharides, such as, without limitation, cross-linked or non-cross-linked: heparin, alginate (alginic acid), guar gum, carboxymethylcellulose (CMC), hyaluronic acid, pullulan, carrageenan, pectin, acid modified chitosan, xanthan gum, agarose, chitosan, collagen, elastin, cellulose, hyaluronic acid, and gelatin, and a mixture of any of the foregoing. Synthetic and/or natural polymer compositions may be cross-linked by any of a large variety of known crosslinking methods, using any of the large variety of known crosslinkers. For example, gelatin and/or hyaluronan crosslinked with methacrylate to produce methacrylated gelatin and/or hyalyronan, e.g., by photocrosslinking.

Although bioerodible constituents may be preferred, non-bioerodible polymers may be used that either do not erode substantially in vivo or erode over a time period of greater than two years. Compositions such as, for example and without limitation, polytetrafluoroethylene (PTFE), poly(ethylene-co-vinyl acetate), poly(n-butylmethacrylate), poly(styrene-b-isobutylene-b-styrene), and polyethylene terephthalate are considered to be non-bioerodable polymers. Other suitable non-bioerodable polymer compositions are broadly known in the art, for example, in stent coating and transdermal reservoir technologies. The growth scaffolds described herein may comprise a non-erodible polymer composition.

Generally, any type of extracellular matrix (ECM) can be used to produce ECM products to be implanted with the cell structure or sheet as described herein. ECM materials are prepared, for example, from decellularized or devitalized ECM material, that optionally has not been dialyzed. ECM materials are broadly-known, and are commercially-available in many forms, and may be prepared from a natural ECM (tissue), or from an in vitro source wherein the ECM is produced by cultured cells and comprises one or more polymeric components (constituents) of native ECM. ECM can be engineered into a variety of three-dimensional structures (see, e.g., Wade, et al. “Engineering ECM Signals Into Biomaterials” Materials Today, 2012; 15(10):454-459).

In aspects ECM is isolated from a vertebrate animal, for example, from a warm blooded mammalian vertebrate including, but not limited to, human, monkey, pig, cow, horse, or sheep. The ECM may be derived from any organ or tissue, including without limitation, nerve tissue, connective tissue, urinary bladder, intestine, liver, heart, esophagus, spleen, cartilage, meniscus, bone, stomach, or dermis. Tissue for preparation of ECM as described herein may be harvested in any useful manner. The ECM can comprise any portion or tissue obtained from an organ, including, for example and without limitation, and where relevant, submucosa, epithelial basement membrane, tunica propria, etc. The ECM material may take many different forms, though in the context of nerve tissue repair, is a sheet, tube, bundled fiber, cylinder, or nerve-shaped, and affixed in place at the site of implantation using a medically acceptable adhesive or sutures.

As used herein, the terms “extracellular matrix” and “ECM” refer to a natural composition useful for cell growth. ECM is decellularized or devitalized tissue, and is a complex mixture of structural and non-structural biomolecules, including, but not limited to, proteins, glycosaminoglycans, proteoglycans, antimicrobials, chemoattractants, cytokines, and growth factors, such as collagens, elastins, and laminins. In mammals, ECM often comprises about 90% collagen in its various forms. The composition and structure of ECMs vary depending on the source of the tissue. For example, small intestine submucosa (SIS), urinary bladder matrix (UBM), liver stroma ECM, and dermal ECM each differ in their overall structure and composition due to the unique cellular niche needed for each tissue.

As used herein, the term “derive” and any otherword forms or cognates thereof, such as, without limitation, “derived” and “derives”, refers to a component or components obtained from any stated source by any useful method. For example and without limitation, generically, ECM-derived material or an ECM-derived gel refers to a material or gel comprised of components of ECM obtained from any tissue by any number of methods known in the art for isolating ECM. In another example, mammalian tissue-derived ECM refers to ECM comprised of components of a particular mammalian tissue obtained from a mammal by any useful method.

Cell growth scaffolds and nerve guide structures can be formed by any useful method, for example, by solvent casting in a mold, typically with particulate leaching to produce a porous structure, or by 3D printing or dry spinning methods, or by electrodeposition. In one aspect, the structure is cut from a polymeric mesh comprising synthetic and/or natural (e.g., ECM) polymer compositions. In one aspect, for illustrative purposes, a polymeric mesh is electrodeposited, e.g., electrospun onto a target, such as a mandrel, and the resultant structure is shaped, e.g., by cutting, into a suitable shape (see, for example, U.S. Pat. Nos. 8,535,719 B2 and 9,237,945 B2, and United States Patent Application Publication No. 2014/0377213 A1, each of which is incorporated herein by reference in its entirety for their disclosure of electrospinning methods, and variations on electrospun matrices, including synthetic and natural components). While the polymeric mesh may be isotropic or anisotropic, and in one embodiment, the polymeric mesh that is used to prepare the matrix is deposited in an oriented manner, and is therefore anisotropic. Electrospinning and electrodeposition methods are broadly-known, and in electrodeposition, relative movement of the nozzles/spinnerets and target surface, e.g., by deposition onto a rotating mandrel, during electrodeposition can be used to produce an oriented pattern of fibers. As is further broadly-known, more than one polymer composition can be electrodeposited concurrently, or in a desired order, to create a layered structure. Further, solutions comprising other polymers, ECM materials (e.g., ECM gel, or solubilized ECM), cell-culture medium, cells, such as stem cells such as mesenchymal stem cells or adipose-derived stem cells, blood products, and/or therapeutic agents can be electrosprayed onto, or into the formed fiber structure, with variable deposition timing to create optimal layering or release of the soluble fraction.

The composition and structures according to any aspect described herein can also include an active agent, such as, without limitation, one or more of an antiseptic, an antibiotic, an analgesic, an anesthetic, a chemotherapeutic agent, an anti-inflammatory agent, a metabolite, a cytokine, a chemoattractant, a hormone, a steroid, a protein, or a nucleic acid. Therapeutic agents that may be incorporated, by themselves, or in combination with a suitable excipient, into the compositions described herein include, without limitation, anti-inflammatories, such as, without limitation, NSAIDs (non-steroidal anti-inflammatory drugs) such as salicylic acid, indomethacin, sodium indomethacin trihydrate, salicylamide, naproxen, colchicine, fenoprofen, sulindac, diflunisal, diclofenac, indoprofen sodium salicylamide, antiinflammatory cytokines, and antiinflammatory proteins or steroidal anti-inflammatory agents); growth factors; antibiotics, such as, without limitation: acyclovir, afloxacin, ampicillin, amphotericin B, atovaquone, azithromycin, ciprofloxacin, clarithromycin, clindamycin, clofazimine, dapsone, diclazaril, doxycycline, erythromycin, ethambutol, fluconazole, fluoroquinolones, foscarnet, ganciclovir, gentamicin, iatroconazole, isoniazid, ketoconazole, levofloxacin, lincomycin, miconazole, minocycline (e.g., minocycline HCl, or Arestin® microspheres for periodontal use), neomycin, norfloxacin, ofloxacin, paromomycin, penicillin, pentamidine, polymixin B, pyrazinamide, pyrimethamine, rifabutin, rifampin, sparfloxacin, streptomycin, sulfadiazine, tetracycline, tobramycin, trifluorouridine, trimethoprim sulphate, Zn-pyrithione, ciprofloxacin, norfloxacin, afloxacin, levofloxacin, gentamicin, tobramycin, neomycin, erythromycin, trimethoprim sulphate, polymixin B, and silver salts such as chloride, bromide, iodide and periodate.

Any useful cytokine or chemoattractant can be mixed into, mixed with, or otherwise combined with any composition as described herein. For example and without limitation, useful components include growth factors, interferons, interleukins, chemokines, monokines, hormones, and angiogenic factors. In certain non-limiting aspects, the therapeutic agent is a growth factor, such as a neurotrophic or angiogenic factor, which optionally may be prepared using recombinant techniques. Non-limiting examples of growth factors include basic fibroblast growth factor (bFGF), acidic fibroblast growth factor (aFGF), vascular endothelial growth factor (VEGF), hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF), corticotrophin releasing factor (CRF), transforming growth factors α and β (TGF-α and TGF-β), interleukin-8 (IL-8), granulocyte-macrophage colony stimulating factor (GM-CSF), interleukins, and interferons. Commercial preparations of various growth factors, including neurotrophic and angiogenic factors, are available from R & D Systems, Minneapolis, Minn.; Biovision, Inc, Mountain View, Calif.; ProSpec-Tany TechnoGene Ltd., Rehovot, Israel; and Cell Sciences®, Canton, Mass.

Other drugs that may promote wound healing and/or tissue regeneration may also be included.

Although the examples below describe use of a cell sheet in repair of facial nerves, the cell sheets comprising neural crest-derived cells, e.g., dental pulp cells are useful for repair of any peripheral nerves, by placement of the cell sheet adjacent to, e.g., in contact with, a nerve injury, such as an anastomosis, or other nerve graft. Typically, the cell sheet is prepared from a patient's own dental pulp from an extracted tooth, and therefore is autologous, though allogeneic or xenogeneic tissue may be used in aspects or embodiments of the invention.

### Example 1

### Materials and Methods:

Dental pulp cells were isolated from human third molars and expanded in dental pulp stem cell culture medium. Briefly, the DPCs were plated onto a 6-well tissue culture plate at an initial cell density of approximately 20,000 cell/cm². The cells were cultured in a medium containing DMEM high glucose with Glutamax, 20% fetal bovine serum, 100 U/ml penicillin and streptomycin, 50 mg/ml ascorbic acid, and +/−20 ng fibroblast growth factor 2 (FGF2). DPCs formed robust sheets within 10 days of culture. NTF gene expression of DPC sheets was assessed using qRT-PCR. DPC sheets secretome was used to culture SHSY-5Y neurons to test its effect on neurite extension in vitro.

### Results:

DPC sheets were formed that are robust and can be easily handled. DPC sheets expressed high level of BDNF, GDNF, NT3 genes and this effect was enhanced by the addition of FGF2. DPC sheet secretome enhanced neurite extension in SHSY-5Y neurons indicating that DPC sheets have a positive functional effect on neurons.

### Conclusion:

DPC sheets can be formed which secrete neurotrophic factors and enhance neurite extension in neurons. Scaffold-free DPC sheets show great promise as a new therapy to accelerate the regeneration of damaged peripheral nerves and improve functional recovery.

### Example 2

Defect Models:

Three types of defects/treatments that mimic treatment modalities currently used in humans are performed. 1—transect the buccal branch of the rat facial nerve and suture the proximal and distal ends together as an end-to-end anastomosis, 2—remove a 5 mm segment of the buccal branch of the facial nerve, rotate it 180 degrees and reimplant it into the defect site to emulate treatment with an autograft tissue, and 3—mechanically crushing the nerve. To assess the effects of our DPC sheets on nerve regeneration, the defect site is wrapped with a DPC sheet. Defects containing the DPC sheet are compared to defects lacking the sheet and also to unaltered animals.

Outcome Measures:

In rats, the facial nerve controls vibrissae movement, so a measure of nerve regeneration is the return of this motor function. Vibrissae movement is assessed weekly by 3 blinded observers and compared to contralateral movement. Electrophysiology measurements can also be made to assess nerve functionality. Vibrissae movement is expected to return sooner in rats treated with DPC sheets as compared to rats having the surgical treatment alone. Following euthanization of the animals, the facial nerve defect is histologically characterized to evaluate axon extension and myelination. Animals are assessed for up to 12 weeks.

### Example 3

### Materials and Methods

Dental Pulp Cells Isolation and Engineering of Scaffold-Free Cell Sheets:

Dental pulp was isolated from adult human 3rd molars, the teeth were free from carious lesion or other oral infections and they were collected within 24 hours of extraction and transported to lab in phosphate buffered saline with penicillin and streptomycin. A digestion cocktail containing collagenase (3 mg/ml) and dispase (4 mg/ml) (EMD Millipore Corporation and Worthington biochemical, USA) was used to digest dental pulp and obtain a total population of dental pulp cells. The isolated cells were expanded and cultured in growth medium (GM) made up of Dulbecco's Modified Eagle Medium (Gibco Life technologies corporation, USA), 20% fetal bovine serum (Atlanta biological, USA) and 1% penicillin and streptomycin (Gibco Life technologies corporation, USA). Further, upon reaching 80% confluence the cells were passaged and cryogenically stored in liquid nitrogen at −196° C. for future experiments. The multipotency of the isolated DPC was verified by inducing differentiation towards osteogenic lineage; the deposition of mineralized matrix was confirmed through Alizarin red staining.

Dental Pulp Cells from Passage 2-4 were Used to Engineer Cell Sheets:

DPC were plated onto 6 well plate at an initial seeding density of 200,000 cells per well in growth medium supplemented with 50 μg/ml L-Ascorbic acid (Sigma-Aldrich, USA). Cell sheets were formed with/without the effect of Fibroblastic Growth Factor 2 (Peprotech, USA) at a concentration of 5 ng/ml. The dental pulp cells were cultured with medium change once every 2-3 days for 10-12 days to form robust cell sheet that could be easily handled with forceps.

Quantification of Cell Number in Engineered DPC Sheets:

Scaffold-free engineered DPC sheets cultured in growth media +/−FGF2 were rinsed with PBS twice followed by their digestion by incubating in 350 μl of TrypLE express (Gibco Life technologies corporation, USA) at 37° C. for 5-7 minutes. The detached cells were suspended in 1 ml of growth media and they were counted in hemocytometer with trypan blue stain. Cell viability of the dental pulp cells was verified with trypan blue stain; the dead cells absorbing trypan blue stain were excluded from the count.

Histological Characterization of Engineered DPC Sheets:

Engineered DPC sheets were washed with PBS twice and fixed with 10% formalin for 20 minutes and stored in 70% ethanol at 4° C. overnight. The cell sheets were processed in tissue processor (LEICA ASP300S, Leica Instruments GmbH Germany) for standard paraffin embedding. Processed cell sheets were sectioned at 5 microns thickness in microtome (LEICA RM2135 style, Leica Instruments GmbH Germany) and incubated at 60° C. for 15 minutes. The cell sheet sections were processed for Hematoxylin and Eosin (H&E) and immunostaining by deparaffinizing and rehydrating the sectioned samples by series of washes in xylene, ethanol and water.

Hematoxylin and Eosin Stain:

The hydrated samples were stained with H&E (Richard-Allan scientific) by a series of washes in hematoxylin, water, NU-CLEAR™ (acid/alcohol), bluing, water, ethanol, eosin, ethanol, xylene and mounted with xylene based mounting media (Thermo scientific, USA). The images were captured using ZEISS Scope.A1 AXIO microscope.

Immunostaining:

The hydrated sample slides were incubated in 10 mM citrate+0.05% triton X at 60° C. overnight for heat induced epitope retrieval. Further, sample sections were permeabilized with 0.1% triton X and incubated with 5% goat serum for 1 hour. Sections were incubated with either type 1 collagen antibody (Anti-collagen|antibody ab34710, Abcam) at 1:200 concentration overnight at 4° C. or in blocking solution as negative control. The sections were stained with secondary antibody Alexa Fluor 488 (ThermoFischer, USA) at 1:500 concentration for one hour followed by counterstaining with 4′,6-diamidino-2-phenylindole DAPI (Sigma-Aldrich, USA) stain for nuclei at 1:500 concentration. The sections were mounted with aqueous mounting media (Shandon Immu-mount USA). The images were captured using Nikon ECLIPSE Ti microscope and processed in ImageJ software.

Analysis of neurotrophic factor gene expression using reverse transcription polymerase chain reaction (RT-PCR): Scaffold free engineered DPC sheets were harvested after 10-12 days in culture and RNA was extracted using QIAGEN RNEASY® Mini Kit following the manufacturer's protocol. Briefly, the DPC sheet was lysed and homogenized using RLT buffer and mixed with 1 volume of 70% ethanol. This was followed with a series of washes and centrifugation with RW1 and RPE buffer before collecting RNA in 30 μl of RNase-free water. The quantity and quality of the RNA was measured using nanophotometer (NANODROP™ One Thermo Fisher, USA). RNA was collected from cell sheets cultured in media containing +/−FGF2. RNA was also isolated from DPC at the time of plating and human embryonic kidney fibroblasts (hek 293t) as control samples.

RT-PCR was performed with TAQMAN™ PCR kit (Applied Biosystems) to analyze the expression of neurotrophic factor genes using primers for human BDNF, GDNF and NT3 (TAQMAN™ Gene Expression Assays) and GAPDH was used as housekeeping gene. The assay was conducted in QUANTSTUDIO™ 6 Flex (Applied Biosystems Life technologies). The obtained data was analyzed and Ct values greater than 35 were considered as negative readings, the fold change for each of the sample was calculated using the ΔΔCt method. First, the ΔCt value was calculated by normalizing the Ct value of each sample gene to that of the housekeeping gene. Then, the difference in the ΔCt values between the experimental and control group was calculated as the (ΔΔCt). The fold change for each sample was calculated as 2(−ΔΔCt).

Detection of Neurotrophic Factor Protein Secretion by DPC Sheets Using Enzyme Linked Immunosorbent Assay (ELISA):

Conditioned medium (CM) was obtained from DPC sheets cultured in growth medium with/without FGF2, the CM was spun down at 2000 rpm for 5 minutes to remove any cellular debris and stored at −800c for future experiments. The uncultured growth media +/−FGF2 was aliquoted and incubated at 370c for 48 hours and stored at −80° C. for future use as control samples. The amount of BDNF, GDNF and NT3 proteins secreted by DPC in the cell sheet was measured by ELISA using Human BDNF PICOKINE™ ELISA Kit EK0307, Human GDNF PICOKINE™ ELISA Kit EK0362 (Boster Biological technology CA, USA) and Human NT-3 ELISA Kit (RayBiotech, USA) respectively. The ELISA assay was performed following manufacturer's protocol, briefly conditioned medium, growth medium and reconstituted proteins were added to the antibody pre coated 96-well plates and incubated at 37° C. for 90 minutes followed by incubation with biotinylated antibody at 37° C. for 60 minutes. The plates were washed with 0.01M PBS for three times and then incubated with Avidin-Biotin-Peroxidase Complex (ABC) at 37° C. for 30 minutes. The plate was washed with 0.01M PBS for five times and incubated with TMB (3,3′,5,5′-tetramethylbenzidine) solution in dark at 37° C. for 20-25 minutes which developed blue color. Further TMB stop solution was added which changed the color to yellow immediately and the plate was read at optical density (O.D) absorbance value of 450 nm in a spectrophotometer (SYNERGY™ H1 microplate reader, BIOTEK USA) within 30 minutes of adding the TMB stop solution. The quantity of secreted proteins (pg/ml) were calculated against standard curves produced using recombinant protein concentrations provided by the manufacturer using linear regression. The experiment was repeated 3 times using cell sheet formed from cells isolated from 3 different individuals. Mean value±standard deviation represent the biological triplicate; average across 3 experiments.

In Vitro Neurite Outgrowth Assay Using SH-SY5Y Neuroblastoma Cells:

Conditioned media (CM) collected from DPC sheets was used to assess the functional effect of the neurotrophic factors secreted by DPC on SH-SY5Y neuroblastoma cells (ATCC CRL-2266). SH-SY5Y neuronal cells were plated on 8-well Poly-L-Lysine coated chamber slide (11,500 cells/well) with culture media containing DMEM/F12 with 10% fetal bovine serum for 24 hours followed by neuronal induction with 10 uM retinoic acid (ACROS organics, USA) for 48-72 hours of incubation (Kolar et al. 2017). Further, neurite formation and extension was induced by treating the neurons with 400 ul of conditioned media +/−FGF2 obtained from DPC sheet or growth media +/−FGF2 as control. The SH-SY5Y neurons were further cultured for 6 days with conditioned/growth media change once every 2-3 days. After 8-9 days of treatment the cells were washed with PBS twice and fixed for 20 minutes with 4% paraformaldehyde (Sigma-Aldrich, USA) prepared fresh. The SH-SY5Y cells were washed with PBS twice and stained for immunostaining with anti-tubulin βIII antibody (Biolegend, USA) and DAPI at concentrations of 1:250. The images were captured with Nikon ECLIPSE Ti microscope.

To validate the potential axon enhancement effect seen by culturing neurons with DPC conditioned media was due to NTFs, TrK B receptor blocker for BDNF, TrK C receptor blocker for NT3 (R&D systems, USA) and neutralizing antibody against GDNF (R&D systems, USA) were added to the conditioned media at a concentration of 5 μg/ml.

Neurite extensions of SH-SY5Y neurons were manually quantified with ImageJ software. Cell extensions twice the length of their cell body were considered as neurite and the longest neurite was measured for each neurite bearing cell. The percentage of neurite positive cells, the longest neurite outgrowth and range of neurite extensions were quantified and reported from conditioned media from three different human dental pulp cells.

Animal Surgery and DPC Sheet Implantation:

Immunocompromised rats aged 4-6 weeks were purchased from Charles River Laboratories (USA) and were housed under standard conditions of alternate light and dark cycle. Before the surgical procedure, rats were anesthetized by intraperitoneal injection of ketamine hydrochloride (40 mg/kg) plus xylazine hydrochloride (5 mg/kg). The surgical site was prepared by trimming the hairs and cleaning with 10% povidone-Iodine solution swabsticks. Skin incision of 1 cm-1.5 cm was placed over the buccal surface anterior to preauricular region and the buccal branch of the facial nerve was exposed. Using microscissors the nerve was separated and released from its underlying fascia and using a 3 mm flat edge non-serrated forceps, 3 mm of buccal branch of facial nerve was crushed for 20 seconds. The crushed nerve was wrapped with the DPC sheet, the transplanted DPC sheet was secured with 9-0 nylon suture at the proximal and distal end of the crush. The skin incision was closed with 4-0 vicryl suture. After the surgery the rats were placed under the warm bag, allowed to recover consciousness and housed with access to food and water. Post-operative analgesia was administered by mixing acetaminophen (1.5 mg/ml) in the drinking water and the rats were observed for normal eating and drinking with active movement. The control animals underwent the same surgical procedure but no DPC sheet was implanted and to mark the crushed site a 9-0 suture was placed around the nerve loosely. The undamaged contralateral side served as the positive control for this experiment and the experiment was carried out for 4 weeks with each group having 6 animals.

Histology and Immunofluorescence Analysis of the Regenerated Nerve:

Four weeks after the surgery, rats were sacrificed by CO2 inhalation and the regenerated nerves were explanted and fixed in 4% paraformaldehyde. The nerve tissue was flash frozen in OCT media and 5 micron sections were cut in cryostat. Routine hematoxylin and eosin staining was done to analyze the general architecture of the nerve.

Further sections were processed for immunofluorescence to detect the regenerated axons. Primary antibody for anti-beta tubulin (1:100) was incubated overnight at 40c and Alexa fluor 488 (1:500) was used as secondary antibody. Nuclei was counterstained by DAPI and the images were captured by NIKON eclipse TE2000-E microscope and processed with imageJ software.

Statistical Analysis: The data is presented as means±standard deviations.

Statistical comparison of the quantified number of cells in cell sheets cultured in +/−FGF2 media was done using Student's t-test. qRT-PCR data was analyzed by comparing the fold change results between treatment and control group using one-way ANOVA with post-hoc Tukey test. ELISA data was analyzed using the final picogram concentrations between treatment and control group by one-way ANOVA with post-hoc Tukey correction. All the statistical tests were done in Graphpad Prism software and statistical difference at p value less than 0.05 was considered significant.

### Results

Engineering DPC Sheets.

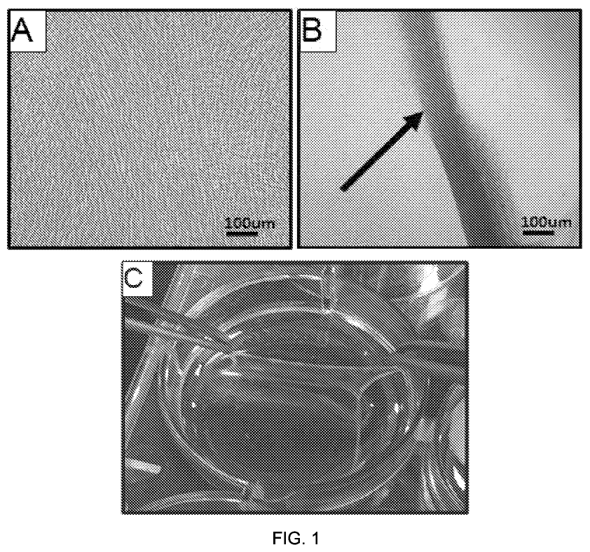

Cell sheets were engineered from the dental pulp cells isolated from pulp of human third molars as shown in FIG. 1.

DPCs when plated at a seeding density of 200,000 cells/well reached to confluence in 2-4 days, and with a further culture for 8-10 days they formed cell sheet which started to detach from the base of the well (FIGS. 1A and 1B). The formed DPC sheets were robust solid tissue structure and able to handle with the forceps (FIG. 1C). The results were consistent with DPC sheets being formed from dental pulp cells isolated from three different patients.

Histological Characterization of DPC Sheets.

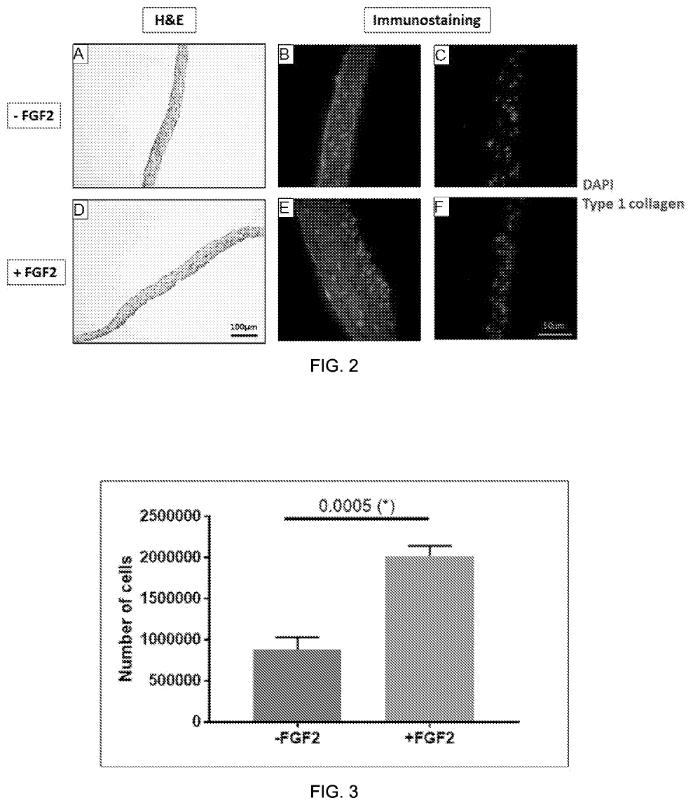

Cell sheets engineered from culturing in the media with/without FGF2 were histologically characterized, the hematoxylin and eosin stain confirmed that the formed cell sheets in both the groups were solid multicellular tissue structure as shown in FIGS. 2A and 2D.

Immunostaining for type 1 collagen confirmed the presence of extracellular matrix in the cell sheets along with the presence of nuclei stained for DAPI as shown in FIGS. 2B and 2E. An intriguing difference in the number of DAPI stained nuclei was observed between the cell sheets formed with FGF2 when compared with the ones without FGF2 in their culture media.

Quantification of Cell Number in DPC Sheet.

The cell number in the DPC sheet cultured with FGF2 contained approximately 2 million cells which was twice the number compared to DPC sheets cultured without FGF2 which had close to 1 million cells as shown in FIG. 3.

The results were consistent for cell quantification procedure which was repeated three times with cells from three different patients. The cell viability verified by the trypan blue stain showed that the cells were almost 100% alive across the 3 experiments. Students t-test showed that the difference noticed was statistically significant with p value less than 0.05.

Reverse Transcription-Polymerase Chain Reaction to Detect NTF Gene Expression.

RT-PCR done with RNA extracted from DPC sheets cultured +/−FGF2, sub confluent DPC and human embryonic kidney fibroblasts showed the expression of NTF genes BDNF, GDNF and NT3.

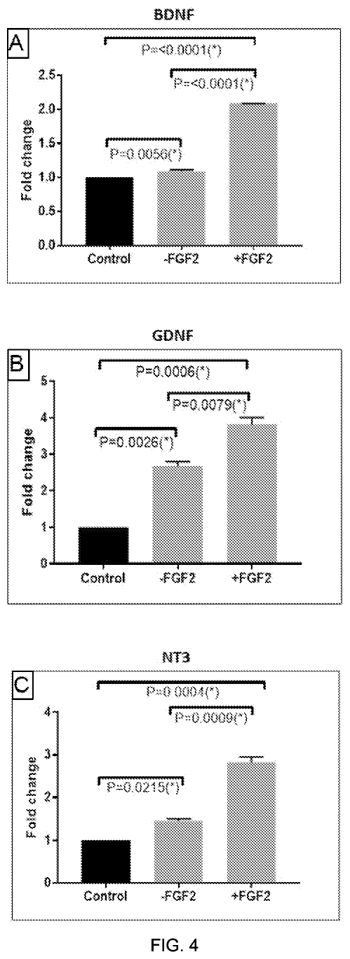

The expression of BDNF gene in the cell sheets cultured in media with FGF2 was upregulated significantly when compared to the other groups without FGF2 and controls as seen in FIG. 4 (A). A similar trend of upregulation for the genes GDNF and NT3 was also noticed as shown in FIG. 4 (B) and 4 (C). One-way ANOVA analysis with tukey's post hoc test showed the difference to be statistically significant between the groups at a P value less than 0.05.

Enzyme Linked Immunosorbent Assay (ELISA) to Detect the Concentration of Proteins.

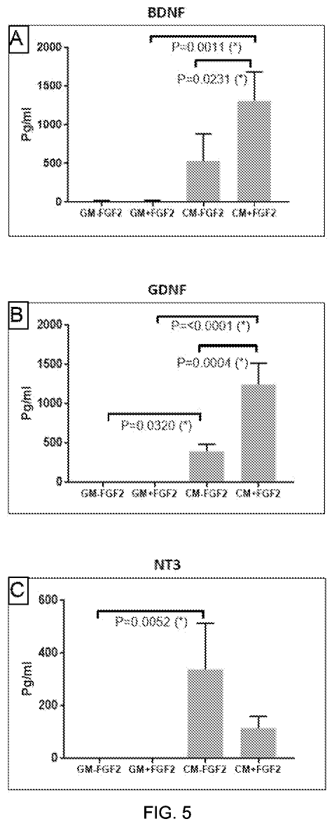

ELISA analysis done from the conditioned media collected from DPC sheet and growth media as negative control showed that the DPCs secrete BDNF, GDNF and NT3 proteins as shown in FIG. 5 (A-C).

The concentration of proteins BDNF and GDNF was increased in the conditioned media with FGF2 (CM+FGF2) when compared to conditioned media without FGF2 (CM-FGF2) and the difference was statistically significant as seen in FIGS. 5A and 5B. The concentration of NT3 protein was found to be more in CM-FGF2 group in comparison with CM+FGF2 as seen in FIG. 5C. The presence of proteins was not detected in control samples that is growth media with/without FGF2.

Functional Evaluation of DPC Sheet Conditioned Media on SH-SY5Y Neuroblastoma Cells.

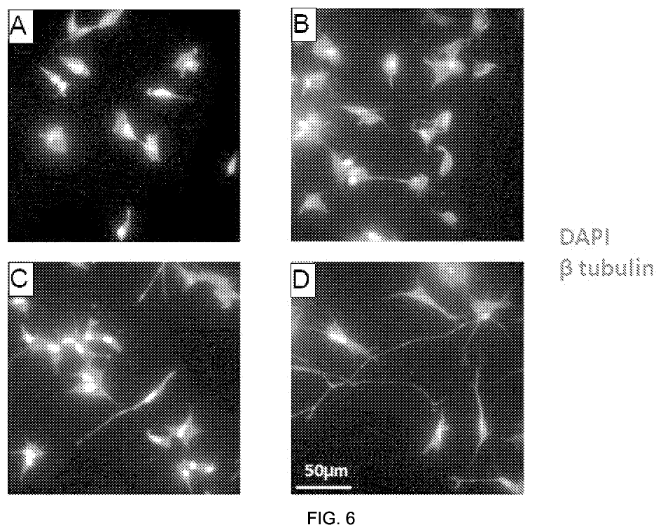

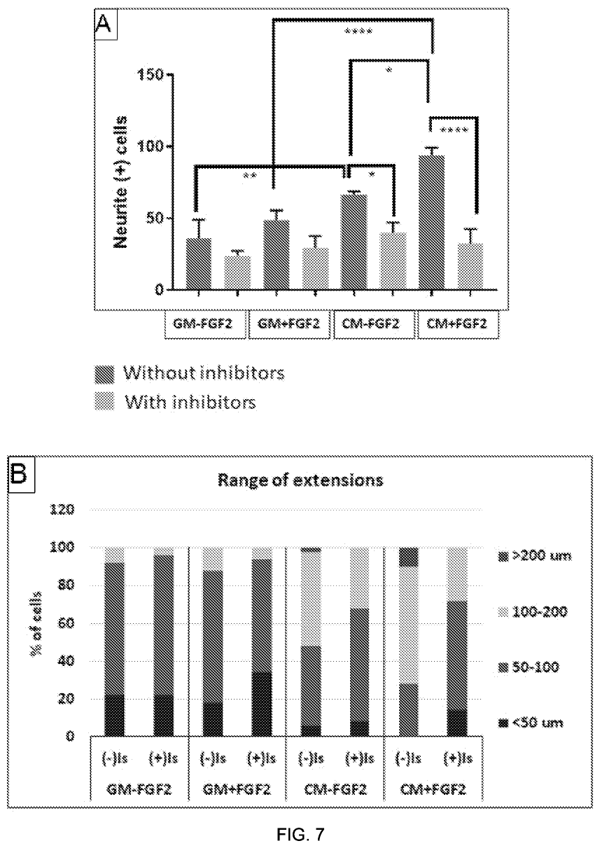

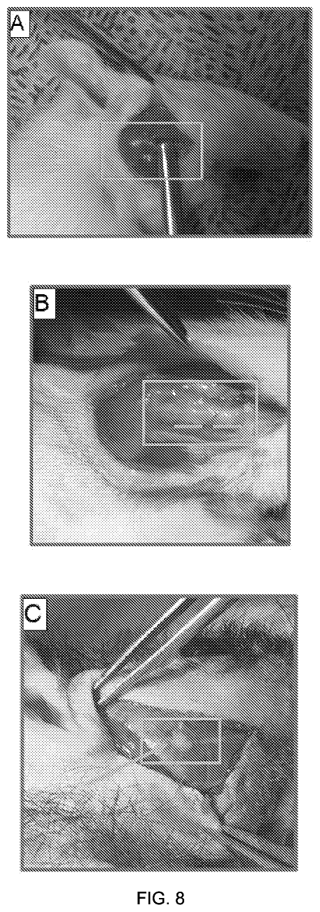

Culturing SH-SY5Y neurons with growth media +/−FGF2 and conditioned media +/−FGF2, we found that the neurite extensions were evident when cultured with the conditioned media from DPC as shown in FIGS. 6C and 6D. The effect was more pronounced and enhanced with CM+FGF2 where SH-SY5Y neuronal cells had increased neurite positive cells with majority of the neurites in the range of 100-200 microns and few more than 200 microns when compared with CM-FGF2 where the neurite length extensions were approximately equally distributed in the range of either 50-100 microns or 100-200 microns and some neurite extensions were less than 50 microns as shown in FIGS. 7A and 7B. When SH-SY5Y cells were cultured with the growth media majority of the neurite extensions were in the range of 50-100 microns with some less than 50 microns. The neurite forming effect of SH-SY5Y neuronal cells was validated with the addition of inhibitory blockers to the conditioned media or growth media.

The neurite formation and extension effect was reversed as there was significant decrease in the neurite positive cells and length of neurite extensions formed as shown in FIGS. 7 (A) and 7 (B).

Evaluation of Nerve Regeneration in Rats.

To evaluate if DPC sheets were able to enhance nerve regeneration, in vivo study was performed above. The buccal branch of the facial nerve in rats was exposed and crushed for 20 seconds (FIG. 8 (A)) and the DPC sheet was implanted in the treatment group (FIG. 8 (B)). After 4 weeks, the surgical site was revisited to explant the regenerated nerve and the implanted DPC sheet was seen to be present and integrated around the nerve as shown in FIG. 8 (C) in the animals of treatment group.

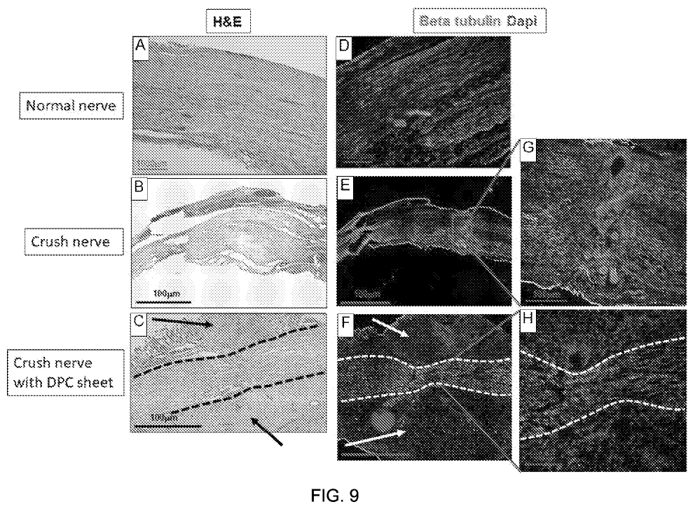

Hematoxylin and eosin (H&E) staining of normal healthy nerve can been seen in FIG. 9 (A), and FIG. 9 (B) shows continuous axon extension in healthy nerves as seen by beta tubulin immunostaining. H&E of the crushed nerves 4 weeks after injury without and with the addition of a cell sheet can be seen in FIGS. 9 (B) and (C), respectively. Beta tubulin immunostaining reveals that untreated crushed nerves did not have continuous axon extension across the defect site (Figure (E) and (G)). Whereas the crushed nerve treated with DPC sheet showed the presence of more continuous axons across the defect (FIGS. 9 (F) and (H)). The DPC sheet can still be seen wrapped around the nerve following the 4 week implantation (FIGS. 9 (C) and (F)).

In conclusion, we have created a scaffold-free cellular sheet that expresses neurotrophic factors for nerve regeneration. We have developed a novel method to generated generate a scaffold-free cell sheet comprising of NTF-expressing dental stem cells and their endogenous matrix. We have data showing that the cells in these sheets are expressing NTFs at the gene level and that these cell sheets induce neuritogenesis in a neuronal cell line in vitro. We are currently implanting these cell sheets in a rat nerve defect model to assess their regenerative capability in vivo. These cells sheets can be wrapped around nerves repaired using standard methods to accelerate healing and enhance motor recovery, or could be wrapped around already commercially available nerve conduits.

The following numbered clauses describe various aspects or embodiments of the present invention:

Clause 1: A method of producing neurite outgrowth in a neuron, comprising: culturing neural crest-derived cells to produce one or more neurotrophic factors; and exposing neural tissue comprising a neuron to one or more neurotrophic factors produced by the cultured neural crest-derived cells for a time sufficient to produce neurite outgrowth from a neuron, thereby producing neurite extension from one or more neurons of the neural tissue.
Clause 2: The method of clause 1, wherein the neural crest-derived cells are cultured to produce a solid tissue structure.
Clause 3: The method of clause 2, wherein the solid tissue structure is a sheet.
Clause 4: The method of clause 1, wherein the neural crest-derived cells are cultured to confluence or post-confluence, to produce a solid tissue structure.
Clause 5: The method of any one of clauses 2-4, wherein the neural tissue is exposed to the one or more neurotrophic factors produced by the tissue structure by implanting the tissue structure adjacent to neural tissue in a patient.
Clause 6: The method of clause 5, wherein damaged nerve tissue is contacted with the tissue structure at a site of nerve damage.
Clause 7: The method of clause 6, wherein the damaged nerve tissue is a peripheral nerve.
Clause 8: The method of clause 6, wherein the damaged nerve tissue is a facial nerve.
Clause 9: The method of clause 6, wherein the damaged nerve tissue is damaged from trauma.
Clause 10: The method of clause 6, wherein the damaged nerve tissue is damaged from disease.
Clause 11: The method of clause 6, wherein the damaged nerve tissue is damaged from surgical injury, or from surgical repair, such as an anastomosed peripheral nerve or a facial nerve that is surgically repaired after trauma or disease.
Clause 12: The method of any one of clauses 2-4, wherein the neural tissue is exposed to the one or more neurotrophic factors produced by the tissue structure by culturing the tissue structure adjacent to the neural tissue.
Clause 13: The method of any one of clauses 2-4, wherein the neural tissue is exposed to the one or more neurotrophic factors produced by the tissue structure by culturing the neural tissue in media conditioned by the tissue structure.
Clause 14: The method of any one of clauses 1-13, wherein the neural crest-derived cells are autologous to the neural tissue.
Clause 15: The method of any one of clauses 1-14, wherein the neural crest-derived cells are dental pulp cells.
Clause 16: The method of any one of clauses 1-15, wherein the neural crest-derived cells are prepared by culturing neural crest-derived cells on a surface, and growing the cells to confluence or to post-confluence to form the tissue structure.
Clause 17: The method of clause 16, wherein the surface is planar and the tissue structure is a sheet of cells.
Clause 18: The method of any one of clauses 1-17, wherein the neural crest-derived cells are a population of cells comprising stem cells and/or progenitor cells.
Clause 19: The method of any one of clauses 1-18, wherein the neural crest-derived cells are cultured in the presence of fibroblast growth factor 2 (FGF2).
Clause 20: The method of clause 19, wherein the FGF2 is present in culture medium in a concentration ranging from 0.25 ng/ml to 25 ng/ml.
Clause 21: The method of any one of clauses 1-20, wherein the neural crest-derived cells are cultured in the presence of ascorbic acid.
Clause 22: The method of any one of clauses 1-21, wherein the nerve cells are grown or cultured on or in a nerve guide, a cell growth matrix, decellularized tissue, or an ECM material.
Clause 23: A method of repairing damaged nerve tissue in a patient, comprising implanting cultured neural crest-derived cells, such as cell populations comprising neural crest-derived stem cells and/or neural crest-derived progenitor cells, adjacent to a site of nerve tissue damage in the patient, thereby causing neurite outgrowth in the nerve tissue.
Clause 24: The method of clause 23, wherein the neural crest-derived cells are autologous to the patient.
Clause 25: The method of clause 23 or 24, wherein the neural crest-derived cells are dental pulp cells.
Clause 26: The method of any one of clauses 23-25, wherein the neural crest-derived cells are a population of cells comprising stem cells and/or progenitor cells.
Clause 27: The method of any one of clauses 23-26, wherein the neural crest-derived cells are cultured in the presence of fibroblast growth factor 2 (FGF2).
Clause 28: The method of clause 27, wherein the FGF2 is present in culture medium in a concentration ranging from 0.25 ng/ml to 25 ng/ml.
Clause 29: The method of any one of clauses 23-28, further comprising culturing the neural crest-derived cells in the presence of ascorbic acid.
Clause 30: The method of any one of clauses 23-29, wherein the neural crest-derived cells are cultured ex-vivo and grown to confluence or to post-confluence to form a cell structure.
Clause 31: The method of any one of clauses 23-29, wherein the neural crest-derived cells are cultured ex-vivo and grown to confluence or to post-confluence on a surface to form a living cell sheet.
Clause 32: The method of clause 31, wherein the damaged nerve tissue is wrapped in the living cell sheet of cultured neural crest-derived cells at the site of nerve damage.
Clause 33: The method of any one of clauses 23-32, wherein the nerve tissue is a peripheral nerve.
Clause 34: The method of any one of clauses 23-32, wherein the nerve tissue is a facial nerve.
Clause 35: The method of any one of clauses 23-32, wherein the nerve tissue damage is from trauma.
Clause 36: The method of any one of clauses 23-32, wherein the nerve tissue damage is from disease.
Clause 37: The method of any one of clauses 23-32, wherein the nerve damage is from surgical injury, or from surgical repair, such as an anastomosed peripheral nerve or a facial nerve that is surgically repaired after trauma or disease.
Clause 38: The method of any one of clauses 23-32, further comprising implanting with the living sheet a nerve guide, a cell growth matrix, and/or decellularized tissue or an ECM material.

While the present invention is described with reference to several distinct embodiments, those skilled in the art may make modifications and alterations without departing from the scope and spirit. Accordingly, the above detailed description is intended to be illustrative rather than restrictive.

1. A method of producing neurite outgrowth in a neuron, comprising:
culturing neural crest-derived cells, such as cell populations comprising neural crest-derived stem cells and/or neural crest-derived progenitor cells, to produce one or more neurotrophic factors; and
exposing neural tissue comprising a neuron to one or more neurotrophic factors produced by the cultured neural crest-derived cells for a time sufficient to produce neurite outgrowth from a neuron, thereby producing neurite extension from one or more neurons of the neural tissue.

2. The method of claim 1, wherein the neural crest-derived cells are cultured to produce a solid tissue structure, such as a sheet.

3. (canceled)

4. The method of claim 1, wherein the neural tissue is exposed to the one or more neurotrophic factors produced by the tissue structure by implanting the tissue structure adjacent to neural tissue in a patient.

5. The method of claim 4, wherein damaged nerve tissue is contacted with the tissue structure at a site of nerve damage.

6. The method of claim 5, wherein the damaged nerve tissue is a peripheral nerve, such as a facial nerve.

7. The method of claim 5, wherein the damaged nerve tissue is damaged from trauma, from disease, from surgical injury, or from surgical repair, such as an anastomosed peripheral nerve or a facial nerve that is surgically repaired after trauma or disease.

8. The method of claim 2, wherein the neural tissue is exposed to the one or more neurotrophic factors produced by the tissue structure by culturing the tissue structure adjacent to the neural tissue, or by culturing the neural tissue in media conditioned by the tissue structure.

9. The method of claim 1, wherein the neural crest-derived cells are autologous to the neural tissue.

10. The method of claim 1, wherein the neural crest-derived cells are dental pulp cells.

11. The method of claim 1, wherein the neural crest-derived cells are cultured in the presence of fibroblast growth factor 2 (FGF2), optionally wherein the FGF2 is present in culture medium in a concentration ranging from 0.25 ng/ml to 25 ng/ml, and/or in the presence of ascorbic acid.

12. The method of claim 1, wherein the nerve cells are grown or cultured on or in a nerve guide, a cell growth matrix, decellularized tissue, or an ECM material.

13. A method of repairing damaged nerve tissue in a patient, comprising implanting cultured neural crest-derived cells, such as cell populations comprising neural crest-derived stem cells and/or neural crest-derived progenitor cells, adjacent to a site of nerve tissue damage in the patient, thereby causing neurite outgrowth in the nerve tissue.

14. The method of claim 13, wherein the neural crest-derived cells are autologous to the patient.

15. The method of claim 13, wherein the neural crest-derived cells are dental pulp cells.

16. The method of claim 13, wherein the neural crest-derived cells are cultured in the presence of fibroblast growth factor 2 (FGF2), optionally wherein the FGF2 is present in culture medium in a concentration ranging from 0.25 ng/ml to 25 ng/ml, and/or in the presence of ascorbic acid.

17. The method of claim 13, wherein the neural crest-derived cells are cultured ex-vivo and grown to confluence or to post-confluence to form a living cell structure, and optionally wherein the neural crest-derived cells are grown to confluence or to post-confluence on a surface to form a living cell sheet.

18. The method of claim 17, wherein the damaged nerve tissue is wrapped in a living cell sheet of cultured neural crest-derived cells at the site of nerve damage.

19. The method of claim 17, further comprising implanting with the living cell structure a nerve guide, a cell growth matrix, and/or decellularized tissue or an ECM material.

20. The method of claim 13, wherein the nerve tissue is a peripheral nerve, such as a facial nerve.

21. The method of claim 13, wherein the nerve tissue damage is from trauma, from disease, or from surgical injury, or from surgical repair, such as an anastomosed peripheral nerve or a facial nerve that is surgically repaired after trauma or disease.
